(19) United States
(12) Patent Application Publication
(10) Pub. No.: US 2015/0004142 A1
(43) Pub. Date: Jan. 1, 2015

(54) INCORPORATION OF CULTURED BILBERRY CELLS IN COSMETICS, DIETARY SUPPLEMENTS, AND/OR FUNCTIONAL FOODS

(71) Applicant: DianaPlantSciences, Inc., Portland, OR (US)

(72) Inventors: Colby G. Caldwell, Oregon City, OR (US); Sung-Yong H. Yoon, Lake Oswego, OR (US)

(21) Appl. No.: 14/316,495

(22) Filed: Jun. 26, 2014

Related U.S. Application Data
(60) Provisional application No. 61/839,808, filed on Jun. 26, 2013.

(51) Int. Cl. A61K 36/45; A23L 1/30

(52) U.S. Cl. CPC A61K 36/45; A23L 1/3002; A23V 2002/00
USPC 424/93.7

(57) ABSTRACT

Compositions include cultured bilberry cells or extracts thereof mixed with a cosmetic component or a food component to yield cosmetics, dietary supplements, and/or functional foods. The cultured bilberry cells or extracts can have high levels of polyphenols with little or no anthocyanins. The polyphenol fraction from the cultured bilberry cells is unique compared to the polyphenol fraction from the tissues of a traditional bilberry plant. The cultured cells have high levels of natural flavonols, flavan-3-ols and procyanidins, but are notably lacking in anthocyanins and chlorogenic acid.

### CROSS-REFERENCE TO RELATED APPLICATIONS

This application claims the benefit of and priority to Provisional Application No. 61/839,808, filed Jun. 26, 2013, which is incorporated by reference in their entirety.

### BACKGROUND

1. Field of Use

The present invention relates to the production of bilberry cells having anti-inflammatory and antioxidant properties, extracts prepared from these cells, and compositions incorporating the cells or extracts.

2. Related Technology

There exists a significant unmet need for natural anti-inflammatory agents. A healthy inflammatory response is a hallmark of a well functioning immune system. Whether challenged by physical injury, invaded by microorganisms or contaminated by a foreign substance, the human body is able to herald an army of killer cells and defensive compounds to the site of injury. Localized inflammation is the result of this response, and in the case of acute injury, it is both effective and appropriate, as long as it is short lived.

When the inflammatory response becomes prolonged, inflammation begins to have serious adverse effects. Over time, inflamed tissues start to breakdown and function abnormally. So serious is this loss of function that chronic inflammation is often considered the root of chronic disease. Chronic or uncontrolled inflammation can lead to a wide range of pathologies, including sepsis, cancer, arthritis, neurodegenerative disease, obesity, diabetes, and atherosclerosis. (Glass C K, Saijo K, Winner B., Marchetto M. C., Gage F. H. Mechanisms underlying inflammation in neurodegeneration. Cell. 2010, 140:918-934.; Medzhitov R. Origin and physiological roles of inflammation. Nature 2008, 454:428-435.; Grivennikov S. I., Greten F. R., Karin M. Immunity, inflammation, and cancer. Cell 2010, 140:883-899.; Hotamisligil G. S. Endoplasmic reticulum stress and the inflammatory basis of metabolic disease. Cell 2010, 140:900-917.) Given that grim list, it is clear that agents capable of countering chronic inflammation and its adverse effects have potentially great value.

Yet inflammation is only one factor contributing to chronic disease; another causal factor is oxidation. Indeed, these two factors are linked since oxidative stress is a primary trigger of the inflammatory response. Thus any discussion of inflammation is incomplete without also considering the role of oxidation. An increase in reactive oxygen species (ROS), along with a concomitant disruption in redox balance, leads to a state of chronic inflammation. Evidence increasingly points to the emerging theory of oxidation-inflammation as the main cause of aging and chronic disease. The term oxi-inflamm-aging has been coined to describe this process, and there is an increasing demand to find remedies for it (De la Fuente M., Miguel J. An update of the oxidation-inflammation theory of aging: the involvement of the immune system in oxi-inflamm-aging. Curr Pharm Des. 2009, 15(26):3003-26).

Bilberry (Vaccinium myrtillus) is one of several plants with a long history of medicinal use. Best known for the anthocyanin content of its berries, bilberry plants (particularly the leaves) also contain flavonols, catechins, procyanidins and phenolic acids.

However, Bilberry is difficult to grow and is therefore rarely cultivated. The fruit is generally collected from wild plants found during its limited growing season (May through September). Thus, the supply of the berries is unreliable and the berries are available in limited quantities. Moreover, the fruit are softer and juicier than the related blueberry, such that they must be harvested by hand, and are difficult to transport, which contribute to the high cost of the fresh fruit. Also due to the high demand for the ripe fruit, unripe fruits and leaves are not economically viable products to collect, reinforcing the focus of existing commercial bilberry-derived products on anthocyanins rather than the procyanidins typically mostly found in unripe materials.

### BRIEF DESCRIPTION OF THE DRAWINGS

FIG. 1 illustrates compounds produced in high concentration in some cell lines of the cultured cells described herein;

FIG. 2 shows a total ion mass chromatogram of cultured bilberry cells illustrating the extensive number of polyphenol compounds naturally produced in the cultured cells;

FIG. 3 shows a time series of polyphenol production by cultured bilberry cell lines selected for rapid growth and high polyphenol production; and

FIG. 4 illustrates an HPLC profile of cultured bilberry cells.

### DESCRIPTION

### I. Introduction

The present invention relates to cosmetics, dietary supplements, and/or functional foods incorporating cultured bilberry cells or extracts that have high levels of polyphenols with little or no anthocyanins. The polyphenol fraction from the cultured bilberry cells is unique compared to the polyphenol fraction from the tissues of a traditional bilberry plant. The cultured cells have high levels of natural flavonols, flavan-3-ols and procyanidins, but are notably lacking in anthocyanins. Anthocyanins are dark colored compounds that give the natural berry its dark color. Since the cultured cells of the invention produce little or no anthocyanins the cells and extracts thereof are light colored to colorless, while still producing as much or more of beneficial polyphenols as compared to plant tissue. The cultured whole cells and extracts of the present invention have been found to be surprisingly potent anti-oxidation, anti-inflammation and anti-aging compounds when incorporated into dermatological compositions, dietary supplements, and/or functional foods.

The antioxidant and anti-inflammatory agents produced by the cultured bilberry cells are naturally derived compounds that have been shown to produce many of the benefits of synthetic non-steroidal anti-inflammatory drugs (NSAIDs) and corticosteroids, but without the detrimental side effects associated with the long-term use of synthetic drugs.

In addition to being anti-inflammatory agents, many of the polyphenols in the cultured bilberry cells are also powerful anti-oxidants. Polyphenols are characterized by the presence of one or more hydroxy-benzene moieties and are typically derived from the phenylpropenoid biosynthetic pathway. Polyphenols as a class may be further subdivided into phenolic acids, stilbenes, chalcones, flavonoids, anthocyanins and others. Many molecules of this type possess at least some antioxidant and/or anti-inflammatory activity, and while certain compounds have proved to be much more potent than others, it is widely accepted that exposure to polyphenols is linked specifically to the prevention of chronic disease and more generally to moderation of the ageing process (Scalbert A., Manach C., Morand C., Rémésy C., Jiménez L. Dietary polyphenols and the prevention of diseases. Crit Rev Food Sci Nutr. 2005; 45(4):287-306).

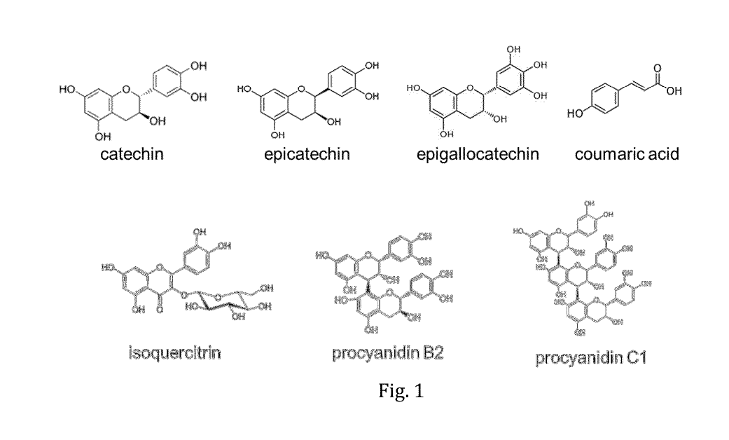

The polyphenol fraction from the cultured cells is made up of a diverse mix of phenolic molecules. The cultured cells produce procyanidins including dimers, trimers, tetramers, etc. In addition to the wide-spread B-type procyanidins, the cultured bilberry cells also produce the doubly-linked A-type procyanidins, which is a class of compounds that is unique to Bilberry (Vaccinium.) Flavonols are also present, mostly if the form of quercetin and kaempferol glycosides. Quercetin glucoside (isoquercitrin) is especially abundant, as is dihydrokaempferol (aromadendrin). Finally, there are the phenolic acids: coumaric acid, caffeic acid and sinapic acid. Examples of specific desired compounds naturally produced in the cultured cells of the present invention may include all or a portion of the compounds shown in FIG. 1, including catechin, epicatechin, epigallocatechin, coumaric acid, isoquercitrin, procyanidins B2, and procyanidins C1.

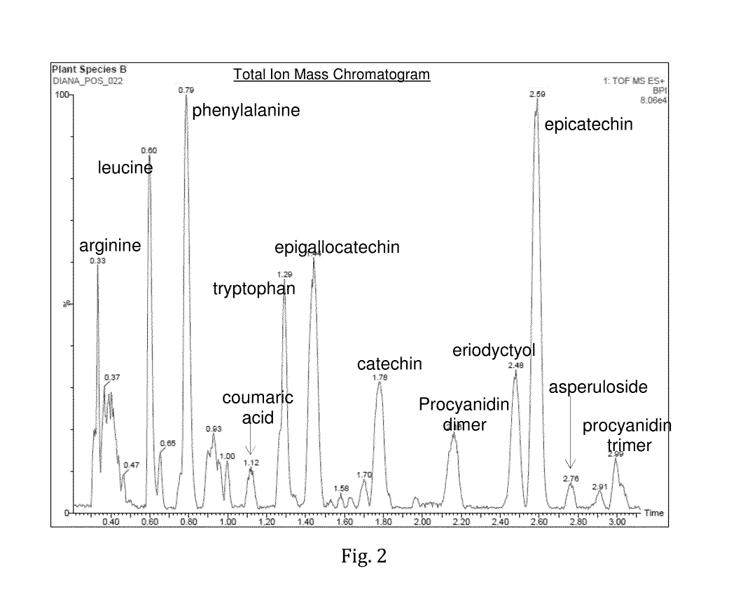

FIG. 2 shows a total ion mass chromatogram of cultured bilberry cells illustrating the extensive number of polyphenol compounds naturally produced in the cultured cells.

Many of the foregoing polyphenols are known to naturally occur in bilberry, but in lower concentrations than is found in the cultured cells described herein. In some embodiments of the invention, the cultured bilberry cells contain at least 10%, 15%, 20%, 25% polyphenols, on a dry weight basis, and/or less than 40%, 35%, 30% polyphenols, and/or within a range of any combination of the foregoing concentrations.

Surprisingly, these concentrations can be achieved with little or no anthocyanin. The anthocyanin concentration may be less than 1.0%, 0.5, 0.1%, 0.01, or even 0.001% on a dry weight basis. It is noteworthy that, the anthocyanins are conspicuously absent from the cultured cells. Most bilberry supplements are dark purple in color and standardized to anthocyanin content, so it is quite unique to have a bilberry product with little or no anthocyanins. The fact that many of the cells are nearly white rather than purple has distinct advantages in cosmetics and food where aesthetics are important.

The cells of the present invention may also be low in chlorogenic acids. In some embodiments, the chlorogenic acid may be less than 1%, 0.1%, or even 0.01% on a dry weight basis. Although not required, minimizing chlorogenic acid may be useful for some people who may suffer from sensitization of allergies from the presence of chlorogenic acid.

In addition, cells grown in cell culture do not express significant quantities of chlorophyll, which is the pigment found in the leaf that gives the leaf its dark green color. Surprisingly the cultured cells have a metabolite profile that is similar to the leaf, even though a cultured cell is very different from a leaf and does not for example have chlorophyll. The amount of chlorophyll may be less than 0.1%, 0.01%, or 0.001% on a dry weight basis or substantially free of chlorophyll.

The unique polyphenol profile including large quantities of anti-inflammatory and anti-oxidant compounds similar to a native leaf, while not producing anthocyanins, chlorophyll, and/or chlorogenic acid has shown to be highly advantageous when incorporated into cosmetic compositions, dietary supplements, and/or functional foods. Skin care formulations including products of the cultured bilberry cells can be applied to the skin to reduce swelling, redness and irritation. The compositions can inhibit the deleterious effects of free-radical induced oxidation to the skin. The cultured bilberry cells can also be formulated into dietary supplements or functional foods with anti-inflammatory and/or antioxidant properties.

The cultured bilberry cells of the present invention can produce polyphenols with little or no anthocyanins in concentrations that are typically 50% or more greater than other cell culture lines known to be high in polyphenols (such as cocoa). Moreover, these results can be achieved in a shorter period of time and using less carbohydrate than other cell lines. Bilberry cells harvested at completion of a growth phase, such as in less than or equal to 10, 9, 8, 7, or 6 days can typically have three times the polyphenols as a cultured cocoa cell line in the same phase of growth and under the same or similar growth conditions. While production of polyphenols in cocoa cell lines may be optimized by adding supplemental glucose near the end or after the end of a growth phase (e.g., after 5-7 days), the bilberry cultured cells have been found to still outperform cultured cocoa cells by a significant margin (e.g., 50%) with respect to polyphenol production when producing high concentrations of polyphenols (e.g., as described herein). For example, cultured bilberry cells may have greater than 30% polyphenols on a dry weight basis when harvested from a culture still in a growth phase. These results are surprising and unexpected.

### II. Production of Cultured Cells

The present disclosure relates to cell culture of Vaccinium myrtillus that are configured to grow in suspension culture in a liquid medium. The cells are derived from one or more Vaccinium myrtillus plant parts. Friable callus can be initiated from hypocotyls, cotyledons, leaves, stems sections roots and the like. The plant tissues can be sterilized by washing and treating the cells with a suitable agent. Seeds can be germinated by suspension in agarose and plated onto plates. For example, the callus may be germinated on Murashige and Skoog medium (4.43 g/L) with agar under a 16 hour light and 8 hour dark photoperiod at 23° C. First signs of callus formation generally occurs a couple of weeks after plating.

The callus cells are selected for subculturing by analyzing the callus and selecting the cells that exhibit desired or superior properties when incorporated into dermatology compositions, dietary supplements, and/or functional foods. For example, callus may be selected for high growth rate, low anthocyanin production (e.g., colorless), and/or high procyanidins production.

Cell suspensions can be created by introducing fresh seedling callus into a liquid medium and agitating the mixture in a shaker. To establish the cell culture, the spent medium may be removed and fresh medium added periodically (e.g., every week for 2 subcultures).

The growth of cells may be estimated by the rate of carbohydrate consumed by measuring the delta of refractive index (RI) (as measured by degrees of BRIX (i.e., % BRIX)) of the medium. If the RI is less than or equal to half of the initial RI of the medium, fresh medium can be added to the cells. If the RI is greater than half, fresh medium can be added after 2 weeks. The subcultures may be transferred weekly or biweekly as deemed necessary.

Cultures that form as either granular or fine suspension of cells are retained, while cultures that do not form suspension cultures are discarded. Packed cell volume (PCV) and RI may be recorded at each subculture to measure cell growth. Sustainable stable suspensions may be obtained within 6 subcultures of initiating suspensions from callus.

Cell culture productivity increases as a function of the rate of cell growth and the density at which cell growth stops. To determine the optimal inoculation density, suspension cultures of Vaccinium myrtillus cells may be initiated with an inoculum size yielding a starting cell density of 12.5%-15% packed cell volume (“PCV”) and 25% PCV and allowed to grow for 7 days. Cell selection can be used to capture cultures that reached a desired PCV or more within 7 days or less (a rapidly growing cell culture). Cultures that take longer than the desired time, are discarded. Preferably the cultures are grown to a PCV of at least 30%, 40%, or 50% within 7 days.

After optimization of growth, the desired production of polyphenol production may be achieved by changing media formulation and using additional criteria for cell selection. The liquid media may be optimized by adjusting carbohydrate level to maintain cultures without nutrient starvation. In some embodiments, the formulation may be formulated with 30 g/L of sucrose to avoid sugar starvation of the cells. In some embodiments, the production values of procyanidins may increase from about 1-2 g/L of PCV at 20 g/L sucrose to about 3-7 g/L of PCV at 30 g/L sucrose. In some embodiments, the carbohydrate concentration may be at least 20 g/L, 30 g/L, 40 g/L, 50 g/L or 60 g/L.

The cells are adapted to produce high concentrations of polyphenols and/or procyanidins and essentially no anthocyanin. Preferably, at least 12.5%, 15%, 20%, 25%, 30%, or more of the dry mass of the plurality of Vaccinium myrtillus cells is comprised of polyphenols. In some embodiments, the polyphenols may be an anti-oxidant and/or anti-inflammatory compound selected from the group (−)-epicatechin, (+)-catechin, procyanidins, quercetin, isoquercetin (quercetin 3-O-glucoside), quercetin 3-O-arabinose, naringenin, or combinations of these. In some embodiments, at least 50%, 60%, 70%, 80%, or 90% of the polyphenolic compounds may be an anti-inflammatory and/or anti-oxidant compound. Preferably, at least 7.5%, 10%, 15%, 20%, or more of the dry mass of the plurality of Vaccinium myrtillus cells is comprised of procyanidins.

It is also preferred that the mass of cells is essentially free of anthocyanins. For example, it is preferred that the dry mass of the plurality of Vaccinium myrtillus cells includes less than 0.5%, 0.1%, 0.01%, 0.001%, or less anthocyanin.

In one embodiment, the method of increasing growth of Vaccinium myrtillus cells in suspension cell culture further includes selecting suspension cell cultures having increased polyphenol and procyanidin accumulation in response to increased sugar concentration in the liquid medium. The increased sugar can be provided near the end of a growth phase in a cell culture or provided in a second stage that follows the growth phase. In one embodiment the sugar concentration may be increased by at least 5, 10, 15, 20, 25, or 30 g/L of cell culture media. The initial concentration of sugar (i.e., during the growth phase) may be less than 20 g/L of cell culture media. The increased concentration of glucose may be at least 25, 30, 40, 50, or 60 g/L and/or less than 100, 80, or 60 g/L or within a range of the foregoing upper and lower concentrations. In one embodiment, the sugar concentration in the liquid medium includes approximately 30-60 g/L sucrose. In one embodiment, procyanidin accumulation in the cells in suspension culture is increased from about 1-2 g/L of PCV at 20 g/L sucrose to about 3-7 g/L of PCV at 30 g/L sucrose. In one embodiment, polyphenol accumulation in the cells in suspension culture is increased from about 2-4 g/L of PCV at 20 g/L sucrose to about 5-10 g/L of PCV at 60 g/L sucrose.

### III. Products Derived from Cultured Bilberry Cells

The cultured bilberry cells are harvested first by removing the spent cell culture medium. Separation can be achieved using centrifugation or other suitable method. The isolated cells may be washed using a suitable fluid (e.g., water) to remove a portion of residual cell culture media.

The isolated cells may be dried to produce a product that can be stored. In one embodiment the moisture content of the dried cultured cells is less than 25%, 20%, 15%, 10%, or even 5%. In some embodiments, the cells may be freeze dried.

In some embodiments, the isolated cells may be milled into a powder. The median particle size may be less than 500 μm, 300 μm, 150 μm, or 100 μm and/or greater than 2 μm, 5 μm, 10 μm, 50 μm, or 100 μm, or within a range of the foregoing upper and lower particle sizes. The desired particle size may be achieved by controlling the milling time, milling type or configuration, and/or by screening out undesired particle sizes.

In some embodiments, the product is a cell culture extract. The cell culture extract can be obtained by suspending a volume of cells in a suitable solvent for extracting the desired compounds or metabolites. The extraction solvent may include water and/or organic compounds. In one embodiment, the solvent includes acetone, acetic acid, and water. In one embodiment, the solvent includes 70% acetone (v/v) and 0.5% acetic acid (v/v).

The organic compound is preferably a food grade compound such as food grade ethanol. In a preferred embodiment, the extraction solvent is hexane free.

In still yet another embodiment, a method of extracting polyphenols from Vaccinium myrtillus cells in culture is described. The method includes (1) selecting a plurality of Vaccinium myrtillus cells adapted to grow in suspension culture, and (2) extracting polyphenols from the cells using a solvent, wherein at least 10% of a dry mass of the plurality of Vaccinium myrtillus cells is comprised of polyphenols and at least 5% of a dry mass of the plurality of Vaccinium myrtillus cells is comprised of procyanidins.

The crude extracts may be filtered to remove undesired particulates. The extraction solvents may be removed by drying to produce a dried bilberry extract from cell culture. Similar to the cultured cells, the extracts have a unique metabolite profile as compared to the native plant tissues.

### IV. Skin Care Products, Dietary Supplements, and Functional Foods

The present invention relates to dermatology compositions incorporating cultured bilberry cells with high concentrations of polyphenols and low concentrations of anthocyanins. The dermatology compositions may be formulated as a paste, cream, gel, spray, powder, solution, or emulsion. When the composition is formulated as a paste, cream or gel, the composition may include animal oil, plant oil, wax, paraffin, starch, tragacanth, cellulose derivatives, polyethylene glycol, silicone, bentonite, silica, talc, zinc oxide, etc. When the composition is formulated as a powder or spray, the composition may include lactose, talc, silica, aluminum hydroxide, calcium silicate or polyamide powder. In particular, a spray composition may include a propellant such as chlorofluorohydrocarbon, propane/butane or dimethyl ether.

For a solution or emulsion, the composition may include a solvent, solubilizer, or emulsifier. Examples include, water, ethanol, isopropanol, ethyl carbonate, ethyl acetate, benzyl alcohol, benzyl benzoate, propylene glycol, 1,3-butylglycol oil, glycerol aliphatic ester, polyethylene glycol or fatty acid ester of sorbitan may be used.

For a suspension, the composition may include a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, and the like.

For a surfactant-containing cleanser, the composition may include aliphatic alcohol sulfate, aliphatic alcohol ether sulfate, sulfosuccinic monoester, isethionate, imidazolinium derivatives, methyl taurate, sarcosinate, fatty acid amide ether sulfate, alkyl amidobetaine, aliphatic alcohol, fatty acid glyceride, fatty acid diethanolamide, vegetable oil, lanolin derivatives, ethoxylated glycerol fatty acid ester, etc. may be used as a carrier. The dermatology composition may also include common adjuvants such as antioxidant, stabilizer, solubilizer, vitamin, pigment and/or fragrance.

The dermatological composition may include cultured bilberry cells in a concentration of greater than and/or equal to 0.01%, 0.1%, 0.5% or 1% by weight or less than or equal to 20%, 10%, 5%, or 1% by weight or within a range thereof.

The dermatological composition may include an extract of cultured bilberry cells in a concentration of at least 0.0001%, 0.001%, 0.01%, or 0.1% by weight and/or less than 10%, 5%, 1%, or 0.1% by weight or within a range thereof.

There is a range of practical applications in which the anti-inflammatory and antioxidant properties of bilberry polyphenols can be used. The dietary supplement and functional foods market is a rapidly growing commercial sector with a high demand for disease-fighting and anti-ageing products. The properties of the cultured bilberry cells that result in beneficial results for the skin can also have beneficial results when included in dietary supplements and functional foods, which come into contact with the sensitive tissues of the digestive track. These tissues can also experience inflammation, and therefore benefit from the cultured bilberry cells of the invention or the extracts thereof.

Dietary supplements are products taken by mouth that include a dietary component (i.e., “dietary ingredient”) intended to supplement the diet. The dietary components may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders.

The cultured plant cells can be incorporated into any food product. Examples of types of foods into which the plant cells may be incorporated include diary, fruit, vegetable, meat, or dessert. The plant cells can be incorporated into raw cooking materials (e.g., flour) or added as a separate ingredient in making a finished food produced (e.g., a baked good). The food component can be of a variety of different types and forms. For example, plant cells may be combined with food component such as, but not limited to, pancakes, cookies, salad dressing, smoothie, milk, scones, chips, yogurt, cheese, vegetables, beans, eggs, bread, cereal, pasta, or flour in various different forms. In yet another embodiment, the heterotrophic cells may be incorporated into a nutritional supplement such as a health tablet.

The cultured plant cells are typically mixed with a food component as a dry powder or granular biomass. For example, powdered plant cells can be mixed with flour or used as a flour in foods such as baked goods or mixed with drinks such as milk or juice or added to liquids or suspensions such as yogurt or incorporated into a pasta (e.g., extruded with the batter to make pasta). A ground or unground granular plant cell may be included in granola bar or cereal.

In some embodiments the food component may also include a powder or granular component. In some embodiments the median particle size of the food component may be less than 150 μm, 100 μm, 50 μm, or 10 μm and/or greater than 0.5 μm, 1 μm, or 5 μm, or within a range of the foregoing upper and lower sizes.

The cultured plant cells may be intimately mixed with the food component as when combined with a flour or baked into a food product. Alternatively, the mixture of the plant cell and the food component maybe more macro. For example, plant cell flakes may be mixed with traditional flakes (e.g., corn flakes derived from cultivated corn). In one embodiment, the food component may be flour, oil, sugar, a dairy product, a fruit, a meat, an herb, and/or a spice.

The amount of the cultured plant cells included in the food products may vary greatly. However, sufficient plant cell is included to have a significant impact on the nutrition or other desired property of the food product. In some embodiments, the amount of the cultured plant cells is at least 1%, 5%, 10%, or 20% by weight and/or less than 80%, 50%, 30%, 15% or 5% by weight of the food product and/or within a range of the foregoing upper and lower values.

In some embodiments, the cultured plant cells may be used as a lightening agent. When used as a lightening agent, the cultured plant cells are sufficiently devoid of pigments (e.g., anthocyanins) that when added to a dermatological component, functional food component, or dietary supplement component, the cultured plant cells dilute the total pigment concentration, thereby lightening the biologically compatible compositions.

### V. Methods for Selecting Cell Lines with Desired Properties

As mentioned, the cultured cells of the present invention are selected to have desired properties such as high polyphenols and/or particular metabolites, while minimizing production of certain compounds such as anthocyanins, chlorophyll, and/or chlorogenic acids. The following methods provide examples of techniques that can be used to identify desired features in the cell lines being produced, thereby allowing selection of particularly desired cell lines. Cell lines having the desired properties can be selected and growing conditions changed to induce a change in the cell lines until the desired result is obtained.

### Metabolic Profiling of Bilberry Cells

An LC/PDA/MS method was developed to determine the amounts and kinds of secondary metabolites produced by bilberry suspension cells. 10 μl of bilberry cell extract was injected using an autosampler into a Waters 626 HPLC fitted with an Ultra-aqueous C18, 3 μm, 100×2.1 mm column. The mobile phase consisted of a water (solvent A)/acetonitrile (solvent B) gradient each containing 0.1% formic acid. The gradient over 35 minutes was 90% A/10% B to 60% A/40% B in 20 minutes then to 100% B in 30 mins followed by a 5 minute hold at 100% B. Flow rate was 0.3 ml/min. Detection was by a Waters 2996 photodiode array (PDA) detector from 191 to 780 nm followed by quadrupole mass spectrometer detection. The mass spectrometer was a Micromass Quattromicro instrument operating in electrospray positive ionization mode (ESI+) with the quadrupole scanning from 198 to 1980 amu. Using this method a range of polyphenolic secondary metabolites was elucidated from bilberry cells (FIG. 1).

### Methods for Testing Activity

The antioxidant activities of bilberry polyphenols are well known and relatively easy to show experimentally. In-vitro antioxidant assays such as ORAC, FRAP, FCR, TEAC, TRAP and DPPH are widely-used, and the antioxidant capacities of the polyphenols produced in bilberry are well documented (Huang D., Ou B., Prior R. L. The chemistry behind antioxidant capacity assays. J Agric Food Chem. 2005, 23; 53(6):1841-56; Maatta-Riihinen K R, Kahkonen M P, Torronen A R, et al. Catechins and procyanidins in berries of vaccinium species and their antioxidant activity. J Agric Food Chem. 2005, 2; 53(22):8485-91). Evidence for the anti-inflammatory activity of bilberry polyphenols also abounds, and several in-vitro assays have been employed in this context (Triebel S, Trieu H L, Richling E. Modulation of inflammatory gene expression by a bilberry (Vaccinium myrtillus L.) extract and single anthocyanins considering their limited stability under cell culture conditions. J Agric Food Chem. 2012, 12; 60(36):8902-10; Chen J, Uto T, Tanigawa S, Kumamoto T, Fujii M, Hou D X. Expression profiling of genes targeted by bilberry (Vaccinium myrtillus) in macrophages through DNA microarray. Nutr Cancer. 2008; 60 Suppl 1:43-50; Karlsen A, Paur I, Bøhn S K, Sakhi A K, Borge G I, Serafini M, Erlund I, Laake P, Tonstad S, Blomhoff R. Bilberry juice modulates plasma concentration of NF-kappaB related inflammatory markers in subjects at increased risk of CVD. Eur J. Nutr. 2010, 49(6):345-55). The efficacy of anti-inflammatory agents is commonly measured by their ability to lower the levels of inflammatory markers such as the cytokines IL-1, IL-6 and IL-8, TNF-α, and more generally to block the actions of NF-kB.

### Determination of Antioxidant Capacity

ORAC and total phenolic content were used to determine the antioxidant potential of bilberry cells. ORAC value as determined by Brunswick Labs, the industry leader for ORAC testing, was >10,000 umole TE/g. Total phenolic content as determined by the Folin-Ciocalteu assay was >20% PP by dry weight.

### Assay of Activity Against Proinflammatory Cytokines TNF-Alpha and IL1-Beta

Bilberry cell extract was tested in a proinflammatory cytokine inhibition assay. The assay used isolated human monocytes stimulated with lipopolysaccharide, and was designed measure inhibition of TNF-alpha and IL1-beta in the presence of a test substance. Induced TNF-alpha and IL-1beta protein were measured by ELISA. (Osteoarthritis Cartilage. 2002 December; 10(12):961-7) Addition of bilberry cell extract to this assay resulted in a significant inhibition of lipopolysaccharide-induced cytokine production.

### Cell Based Assay Against NF-κB

Bilberry cell extract was tested in a cell-based bioassay for inhibitory activity against NF-κB as a demonstration of the extract's anti-inflammatory activity. The A204 NF-κB cell based bioassay showed that bilberry cell extract lowered levels of NF-κB and led to amelioration of the inflammatory response.

### NF-kB ELISA Assay

The assay uses streptavidin-coated plates with bound NF-κB biotinylated-consensus sequence to capture only the active form of NF-κB. The captured active NF-κB is incubated with a specific NF-κB p65 antibody, which is then detected using an HRP conjugated secondary antibody. The assay is developed with a chemiluminescent substrate and the signal is detected using a luminometer.

### Lipopolysaccharide (LPS) Induced Inflammation Model

LPS-induced inflammation models provide a test system for efficacy studies with therapeutic candidates that aim to reduce and/or eliminate excessive inflammatory response. After administration of LPS, it is possible to measure and characterize the cellular profile of recruited leukocytes as well as measure levels of pro-inflammatory cytokines (TNF-α, IL-1β, IL-6, IL-10, etc.) or inducible nitric-oxide (iNOS). LPS-induced edema provides a useful functional model for characterization of the cytokine modulating activity of bilberry cell extracts.

### Inhibition of IL-1β Expression.

With the stimulus of lipopolysaccharide (LPS), IL-1β is overexpressed and its product is increased by inflammatory signal transduction. Human diploid fibroblast (HDF) cells were treated with LPS and various volumes of bilberry cell extract and HDF cells were collected every 6 hr to quantify IL-1β expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-1β at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-1β was reduced in the samples treated by LPS with bilberry cell extract.

### Inhibition of IL-8 Expression.

With the stimulus of lipopolysaccharide (LPS), IL-8 is over expressed and its product is increased by inflammatory signal transduction. Human diploid fibroblast (HDF) cells were treated with LPS and various volumes of bilberry cell extract and the HDF cells were collected every 6 hr to quantify IL-8 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-8 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-8 was reduced in the samples treated by LPS with both bilberry cell extract.

### Carrageenan-Induced Rat Paw Edema Inflammation Model

Inflammation induced by carrageenan is acute, nonimmune, well-researched, and highly reproducible. Cardinal signs of inflammation—edema, hyperalgesia, and erythema—develop immediately following subcutaneous injection, resulting from action of proinflammatory agents—bradykinin, histamine, tachykinins, complement and reactive oxygen, and nitrogen species. Such agents can be generated in situ at the site of insult or by infiltrating cells. Neutrophils readily migrate to sites of inflammation and can generate proinflammatory reactive oxygen and other species. The inflammatory response is usually quantified by increase in paw size (edema) which is maximal around 5 h postcarrageenan injection and is modulated by inhibitors of specific molecules within the inflammatory cascade. Mouse paw edema was used to test the anti-inflammatory activity of bilberry cell extracts.

### Human Skin Acute Inflammation Assay

Fresh, full thickness skin samples were ethically obtained from cosmetic surgery procedures. The assay was used to assess the anti-inflammatory effects of bilberry cells by adding bilberry cell extract topically or to the media (simulating a systemic presence). Validated assays are available for LPS, PHA and UV light-induced acute inflammation. Supernatants can be analyzed for a large range of cytokines, biomarkers and inflammatory modulators with multiple time point sampling possible from the same biopsy.

### VI. Examples

### Example 1

Example 1 describes a method for obtaining cultured cells with a desired polyphenol profile where the concentration of polyphenols is high based on dry weight, while the concentration of anthocyanins and chlorophyll pigments are low or non-existent.

Fresh Vaccinium myrtillus material such as leaves and berries at various stages of ripening were obtained from Oregon State University in Corvallis, Oreg., USA. The leaves and berries were taken from specimens grown locally in Oregon. After sterilization of the raw material, explants were collected and placed in Petri dishes with various growth media. After weeks of culture, explants started dedifferentiating into callus material. The most proliferative and friable calli were then selected and moved into liquid suspension for further cell selection.

Once suspended in liquid medium, cell lines were repeatedly selected over months for growth characteristics and procyanidins production using a modified version of the Swain and Hillis method and Porter et al. method. The butanol-HCl extraction assay was used to measure the concentration of those procyanidins hydrolyzed in monomers of (−)-epicatechin and cyanidin in the acetone-based extracts of Vaccinium myrtillus; a pink color would appear in correlation to the concentration of monomers and be measured by absorbance at 520 nm. The total polyphenol content of bilberry cell extracts was measured using the Folin-Ciocalteau assay and expressed in gallic acid equivalent.

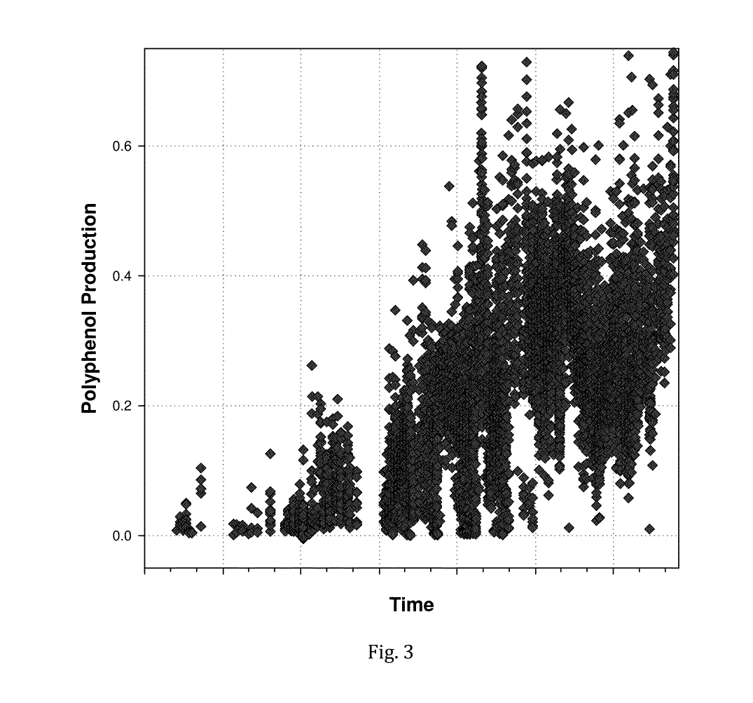

Over the course of three years of selection, the production of polyphenols (and predominantly procyanidins) was increased by a factor of roughly 30× as highlighted in FIG. 3 reproduced below. The existence of some lower data points late in the development process only reveals the various experiments to test for optimal growth and production conditions. Throughout the three years of development, more than 10,000 cell lines were analyzed and ranked using a bioinformatics platform adapted for high-throughput screening and the huge related data generation. FIG. 3 shows a time series of polyphenol production by cultured bilberry cell lines that illustrates the data generated.

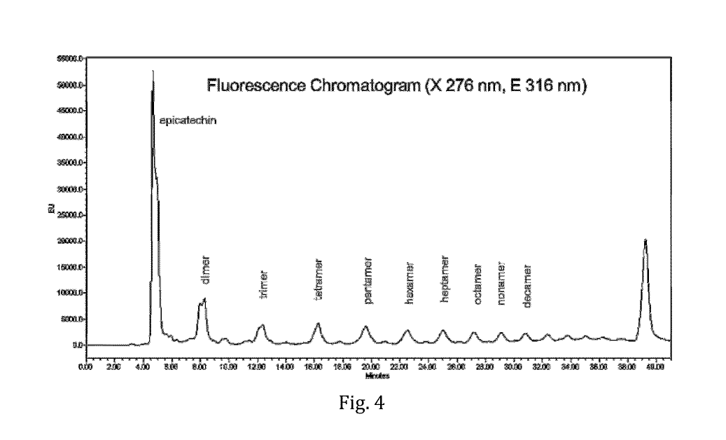

The final product obtained after iterative selection is a cell line that produces about 30% of polyphenol by dry weight, of which a large proportion is made of procyanidins as shown in FIG. 4, which illustrates an HPLC profile of cultured bilberry cells.

The particular cell line was selected for its high growth rate and production of procyanidins. The profile of the metabolic composition is surprising since the cell line was grown from a berry explant, but the metabolic composition is closer to that of bilberry leaves. Chlorogenic acid is surprisingly absent, likely as a compensation of the re-directed flux towards other polyphenols such as procyanidins.

Table 1 below provides a comparison of the polyphenol compounds found in the cultured cell of the invention (first column) as compared to the whole berry (second column), whole leave (third column), and a commercial extract from the berry (fourth column).

TABLE 1

| | Cells | Whole berry | Whole leaf | Commercial Extract |
|---|---|---|---|---|
| Chlorogenic acid | | — | — | — |
| Epigallocatechin | — | | — | |
| Catechin | — | | — | |
| Epicatechin | — | | — | |
| Anthocyanidins | | — | | — |
| Procyanidins | — | | — | |
| Quercetin glucoside | — | — | — | — |

As seen in Table 1, the cultured cells are more similar to the whole leaf than the whole berry or extract in terms of polyphenols, but are still different from the whole leaf with regard to other compounds, such as chlorogenic acid.

### Example 2

### Preparation of Freeze-Dried, Ground Bilberry Cell Powder

Example 2 describes a preparation of a freeze-dried bilberry material. Fresh bilberry cells prepared by the method of Example 1 were harvested from a bioreactor and the cells were washed with DI water to remove residual spent medium. The cells were frozen then freeze-dried to <5% moisture. The dry cells were milled through a 250 μm screen to obtain an off-white powder.

### Example 3

### Preparation of Bilberry Cell Extract

Example 3 describes a method for preparing a bilberry cell extract. Fresh or dried bilberry cells were suspended in a volume of 70% ethanol. The mixture was homogenized then filtered to obtain a clear filtrate. The solid cell cake was re-extracted with another portion of 70% ethanol and again filtered. The combined filtrates were evaporated to dryness to yield crude bilberry cell extract.

### Example 4

### Dermatological Formulation

Example 4 provides a formulation of a cosmetic cream according to one embodiment of the invention.

| No. | Ingredient | Mass % |
|---|---|---|
| 1 | 1,3-Butylene Glycol | 5.0 |
| 2 | Ceramide | 1.5 |
| 3 | Cholesterol | 1.0 |
| 4 | Distilled Water | 72.4 |
| 5 | Glycol Monostearate | 4.0 |
| 6 | Lecithin | 1.0 |
| 7 | Potassium Hydroxide | 0.1 |
| 8 | Stearyl Alcohol | 3.5 |
| 9 | Stearic Acid | 2.5 |
| 10 | Cultured Bilberry cells dried and | 9.0 |
| | ground | |

| | |
|---|---|
| Total | 100 |

### Example 5

### Dermatological Formulation

Example 5 provides an example dermatological formulation according to one embodiment of the invention.

| No. | Ingredient | Mass % |
|---|---|---|
| 1 | Beta sitosterol | 12.0 |
| 2 | Carboxyvinyl Polymer | 0.4 |
| 3 | Ceteareth-20 | 6.0 |
| 4 | Ceramide | 0.1 |
| 5 | Cholesterol | 0.3 |
| 6 | Concentrated Glycerin | 2.0 |
| 7 | DEA-cetyl Phosphate | 0.4 |
| 8 | Distilled Water | 60.15 |
| 9 | Fragrance | 0.15 |
| 10 | Macadamia Nut Oil | 10.0 |
| 11 | Polyglyceryl-2 Oleate | 0.2 |
| 12 | Preservative | 0.2 |
| 13 | Cultured Bilberry cell extract | 8.0 |
| 14 | Xanthan Gum | 0.1 |

| | |
|---|---|
| Total | 100 |

### Example 6

### Functional Dermatological Ointment

Example 6 provides a formulation of a dermatological ointment according to one embodiment of the invention.

| No. | Ingredient | Mass % |
|---|---|---|
| 1 | Boric acid | 1.0 |
| 2 | Zinc oxide | 3.0 |
| 3 | Menthol | 1.0 |
| 4 | White soft paraffin | 25.0 |
| 5 | Cetostearyl alcohol | 20.0 |
| 6 | Sodium laurlyl sulfate | 1.0 |
| 7 | Methyl papaben | 0.1 |
| 8 | Propyl paraben | 0.1 |
| 9 | Triethanolamine | 0.5 |
| 11 | Propylene glycol | 10.0 |
| 12 | Purified water | 31.3 |
| 13 | Cultured bilberry cells dried and | 7.0 |
| | ground | |
| | Total | 100.0 |

1. A biologically compatible composition, comprising,
cultured bilberry cells including at least 10% on a dry weight basis of polyphenolic compounds and less than 1.0% dry weight anthocyanin compounds; and
one or more biologically compatible components including dermatological components or food components, the biologically compatible components mixed with the cultured bilberry cells to yield the biologically compatible composition.

2. The composition of claim 1, wherein the polyphenolic compounds include at least 50% of one or more anti-oxidant and/or anti-inflammatory compounds selected from the group consisting of (−)-epicatechin, (+)-catechin, procyanidins, quercetin, isoquercetin (quercetin 3-O-glucoside), quercetin 3-O-arabinose, naringenin, or combinations of these.

3. The composition of claim 1, wherein the cultured cells have less than 1% chlorogenic acid.

4. The composition of claim 1, wherein the one or more biologically compatible components are dermatological components and the combination of the dermatological components and the cultured cells yield a dermatological suspension, emulsion, paste, gel, cream, lotion, powder, soap, surfactant-containing cleanser, oil, powder foundation, emulsion foundation, wax foundation, or spray.

5. The composition of claim 1, wherein the composition is formulated as a paste, cream, or gel, the composition including animal oil, plant oil, wax, paraffin, starch, tragacanth, cellulose derivatives, polyethylene glycol, silicone, bentonite, silica, talc, or zinc oxide.

6. The composition of claim 1, wherein the one or more biologically compatible components are food components and the combination of the food components and the cultured cells yield a functional food.

7. The composition of claim 6, wherein the food components include flour, oil, sugar, a dairy product, a fruit, a meat, an herb, a spice, or combination thereof.

8. The composition of claim 6, wherein the functional food is a pancake, a cookie, a salad dressing, a smoothie, milk, a scone, a chip, yogurt, cheese, a vegetable product, a bean product, an egg product, a bread, a cereal, or a pasta.

9. The composition of claim 1, wherein the one or more biologically compatible components are food components and the combination of the food components and the cultured cells yield a dietary supplement.

10. The composition of claim 9, wherein the dietary component is a vitamin, a mineral, a botanical, an amino acid, an enzyme, an organ tissue, a glandular, or a metabolite.

11. The composition of claim 9, wherein the dietary supplement is formulated as a tablet, a capsule, a softgel, a gelcap, a liquid, or a powder.

12. The composition of claim 1, wherein the composition includes cultured cells in a concentration in a range from 0.1%-20% by weight.

13. The composition of claim 12, wherein the cultured cells are cells of a non-floral vegetative tissue selected from the group consisting of nodes, internodes, young leaves, mature leaves, stems, roots, and combinations thereof.

14. A method for making a biologically compatible composition, comprising:
culturing isolated bilberry cells in a suspension culture including a liquid media;
harvesting the cultured bilberry cells by removing the liquid media and optionally obtaining an extract from the cultured bilberry cells; and
mixing the harvested cultured bilberry cells or the extract thereof with at least one or more biologically compatible components selected from dermatological components or food components.

15. The method of claim 14, wherein the bilberry cells and liquid media are selected to produce cultured cells having at least 25% polyphenolic compounds, less than 1.0% anthocyanin compounds, and less than 1% chlorogenic acid on a dry weight basis.

16. The method of claim 14, wherein the composition includes cultured cells in a concentration in a range from 0.1%-20% by weight.

17. The method of claim 14, wherein an extract of the cultured bilberry cells is mixed with the biologically compatible components, wherein the extracts are produced by extracting the bilberry cells using an extraction solvent including water, absolute or aqueous lower alcohol containing 1-4 carbons, acetone, ethyl acetate, butyl acetate, dichloromethane (CH₂Cl₂), chloroform, hexane, 1,3-butylene glycol, or combinations thereof.

18. The method of claim 14, wherein the at least one component is a dermatological component that is combined with the cultured bilberry cells or the extract thereof to yield a solution, suspension, emulsion, paste, gel, cream, lotion, powder, soap, surfactant-containing cleanser, oil, powder foundation, emulsion foundation, wax foundation, or spray.

19. A biologically compatible composition, comprising,
a dry powder comprising cultured bilberry cells having at least 20% polyphenolic compounds, less than 1.0% anthocyanin compounds, and less than 1% chlorogenic acid on a dry weight basis.

20. The composition of claim 19, wherein the cultured cells are cells of a non-floral vegetative tissue selected from the group consisting of nodes, internodes, young leaves, mature leaves, stems, roots, and combinations thereof.
